(19) United States
(12) Patent Application Publication
(10) Pub. No.: US 2007/0297986 A1
(43) Pub. Date: Dec. 27, 2007

(54) Screening methods for identifying agents useful for treating or reducing visual impairment in mammals

(75) Inventor: Thomas G. Hampton, Framingham, MA (US)

Correspondence Address: LAHIVE & COCKFIELD, LLP, ONE POST OFFICE SQUARE, BOSTON, MA, 02109-2127, US

(73) Assignee: The Curavita Corporation, Boston, MA (US)

(21) Appl. No.: 11/820,623

(22) Filed: Jun. 20, 2007

Related U.S. Application Data
(60) Provisional application No. 60/815,335, filed on Jun. 21, 2006.

(51) Int. Cl. A61K 49/00; A61K 48/00; A61K 38/17; A61N 5/00

(52) U.S. Cl. 424/9.2; 514/44; 514/12; 607/87

(57) ABSTRACT

Screening methods for identifying agents that affect vision in mammals are provided along with systems which may be used in such methods. Accordingly, methods of the present invention can be used for identifying agents that are useful for treating or reducing visual impairment as well as for identifying agents that result in visual impairment.

### RELATED APPLICATION

This application claims the benefit of priority of U.S. Provisional Patent Application Ser. No. 60/815,335, entitled “Screening Methods For Identifying Agents Useful For Treating Or Reducing Visual Impairment In Mammals,” filed on Jun. 21, 2006, the entire contents of which are incorporated by reference herein.

### FIELD OF THE INVENTION

The present invention relates to screening methods for identifying agents useful for treating or reducing visual impairment and systems for use in such methods.

### BACKGROUND OF THE INVENTION

Visual impairment affects millions of people around the globe every year. Visual impairment may either by congenital or it may occur anytime after birth. Although, visual impairment has been associated with alterations in gait, for example, especially in the elderly where a greater incidence of falling is observed, little is known about which kinematic metrics are affected by visual impairment. Mouse models are widely used to study aging and movement disorders in humans, however, there are no animal models/systems which can be used for investigating the relationship between visual impairment and gait metrics.

Accordingly, there is a need for the development of a system which can be used for investigating the effects of visual impairment on gait.

### SUMMARY OF THE INVENTION

The present invention is based, at least in part, on the discovery of a statistically significant difference in gait metrics, especially the forelimb gait metrics, between visually impaired subjects relative to subjects which are not visually impaired.

In one embodiment, the present invention provides a method for identifying an agent which treats or reduces visual impairment in a subject including: (a) subjecting an experimental vertebrate having visual impairment to the agent; and (b) measuring the forelimb gait metrics of the experimental vertebrate; where an alteration in the forelimb gait metrics in the presence of the agent relative to the forelimb gait metrics in the absence of the agent is an indication that the agent treats or reduces visual impairment.

In another embodiment, the present invention provides a method for identifying an agent which treats or reduces visual impairment in a subject including: (a) subjecting an experimental vertebrate having visual impairment to the agent; and (b) measuring the upperlimb gait metrics of the experimental vertebrate; where an alteration in the upperlimb gait metrics in the presence of the agent relative to the upperlimb gait metrics in the absence of the agent is an indication that the agent treats or reduces visual impairment. In quadruped animals, the upperlimbs may be referred to as the forelimbs. In a bipedal animal that is capable of crawling, such as an infant human, the arms may be referred to as forelimbs. In general, the terms “upperlimbs” and “forelimbs” are used interchangeably herein.

In some embodiments, an experimental vertebrate whose upperlimb gait metrics are measured is a bipedal mammal, such as, for example, a baboon, a chimpanzee or a human.

In one embodiment, an alteration in the forelimb gait metrics in the presence of an agent is a decrease in a statistically significant difference between the forelimb gait metrics of the experimental vertebrate having visual impairment and a vertebrate not having visual impairment.

In another embodiment, the present invention provides a method of identifying an agent which affects vision in an experimental vertebrate comprising: (a) subjecting an experimental vertebrate to the agent; and (b) measuring forelimb gait metrics in the experimental vertebrate, where an alteration in the forelimb gait metrics in the presence of the agent relative to the forelimb gait metrics in the absence of the agent is indicative of an agent which affects vision.

In some embodiments, the experimental vertebrate is administered an agent in vivo. In other embodiments, the eyes of the experimental vertebrate are exposed to the agent.

The agent identified using methods if the present invention as being useful for treating or reducing visual impairment can be any agent which may potentially be useful for treating or reducing visual impairment. In some embodiments, an agent is selected from the group consisting of an organic compound, an inorganic compound, a peptide, an antibody, an antioxidant, a vitamin, an siRNA, photodynamic therapy and laser treatment,

The experimental vertebrate having visual impairment typically includes at least one forelimb and preferably at least three limbs in total. In one embodiment, an experimental vertebrate is a quadrupedal mammal. In some embodiments, the experimental vertebrate having visual impairment is a rodent. Exemplary experimental vertebrates may be selected from the group consisting of a mouse, a rat, a hamster, a chinchilla, a guinea pig, a cat, a dog, or a human, and the like having visual impairment. The visual impairment may either be partial or complete (e.g., total blindness), and may be associated with a neurodegenerative disease or a nervous system disorder such as, for example, Parkinson's disease or multiple sclerosis.

In some embodiments, the experimental vertebrate having visual impairment is placed on a moveable belt track for measuring the gait metrics. The belt track may include one or more impediments and/or visual cues disposed along the belt track. Exemplary visual cues include, but are not limited to, a pattern, a shape, a color or a light stimulus, each of which singularly or together cause the subject to change its posture or gait while walking.

In some embodiments, the gait metrics are measured using ventral plane videography.

The methods of the present invention may be used for identifying agents that may be useful for treating or reducing visual impairment selected from the group consisting of blindness; cataract, myopia, color blindness, macular degeneration, cortical visual impairment, visuospatial disorientation, glaucoma, nystagmus, strabismus, retinoblastoma, retinopathy of prematurity, diabetic retinopathy, and visual impairment associated with Parkinson's disease and multiple sclerosis.

In one embodiment, a gait imaging system used in accordance with the methods of the present invention includes a moveable belt and one or more imaging devices disposed below, along and/or above the moveable belt. In another embodiment, a gait imaging system further includes one or more impediments disposed on the moveable belt. In yet another embodiment, a gait imaging system further includes one or more visual cues disposed in the path of a subject ambulating on the moveable belt. In yet another embodiment, a gait imaging system used in accordance with the methods of the present invention includes a combination of one or more imaging devices disposed below, along and/or above the moveable belt, one or more impediments disposed on the moveable belt, and one or more visual cues disposed in the path of a subject ambulating on the moveable belt.

In one embodiment, the one or more impediments disposed on the belt track are flexibly hinged along the belt track. One or more imaging devices may be selected from the group consisting of a camera, a camcorder, an image capturing device, including the incorporation of a mirror to reflect such images.

### BRIEF DESCRIPTION OF THE DRAWINGS

FIG. 1 is a diagrammatic illustration of a locomotion monitoring apparatus, according to one aspect of the present invention.

FIGS. 2A and 2B are graphs depicting the results of an experiment to measure the gait metrics in visually impaired mice. FIG. 2A depicts the gait metrics for both the right and left hindlimbs and forelimbs for visually impaired (blind) mice walking at 34 cm/s. FIG. 2B depicts the right forelimb gait dynamics in visually impaired (blind) and control (not blind) mice.

FIGS. 3A and 3B depict a schematic of an assay used for measuring the effects of vision on gait, which involves introducing one or more barriers or impediments into the path of a mouse walking on a treadmill belt.

FIGS. 4A and 4B depict ventral views of a mouse through different aspects of stride as well as any impediments, which can be viewed simultaneously using one camera.

### DETAILED DESCRIPTION OF THE INVENTION

The present invention is based, at least in part, on the discovery of statistically significant differences in gait metrics between visually impaired and control subjects, and in particular differences in the forelimb gait metrics of mice with no eyes in comparison to mice with eyes. The present invention provides systems which can be used for measuring differences in forelimb gait metrics, differences in upper limb gait metrics (e.g., in case of bipeds), or differences in hind limb gait metrics, as the case may be, between visually impaired and control subjects and use of such systems in screening methods for identifying agents which are useful in treating or reducing visual impairment.

In one embodiment, a screening method of the present invention includes identifying an agent which treats or reduces visual impairment in a subject by: (a) subjecting an experimental vertebrate having visual impairment to an agent; and (b) measuring the limb gait metrics of the experimental vertebrate; where an alteration in the limb gait metrics in the presence of the agent relative to the limb gait metrics in the absence of the agent is an indication that the agent treats or reduces visual impairment. In one embodiment, the alteration in the forelimb gait metrics includes having no statistically significant difference between the forelimb gait metrics of the experimental vertebrate having visual impairment in the presence of the agent and that of a control vertebrate that is not visually impaired.

In another embodiment, a screening method of the present invention includes identifying an agent which affects vision in a subject by: (a) subjecting an experimental vertebrate to an agent; and (b) measuring the forelimb gait metrics of the experimental vertebrate; where an alteration in the forelimb gait metrics in the presence of the agent relative to the forelimb gait metrics in the absence of the agent is indicative of the agent which affects vision in the subject.

In yet another embodiment, a screening method of the present invention includes identifying an agent which affects vision in a subject by: (a) subjecting an experimental vertebrate to an agent; and (b) measuring the upperlimb gait metrics of the experimental vertebrate; where an alteration in the upperlimb gait metrics in the presence of the agent relative to the upperlimb gait metrics in the absence of the agent is indicative of the agent which affects vision in the subject.

Accordingly, in some embodiments, a screening agent of the present invention may be used for identifying agents which result in visual impairment.

The term “agent,” as used herein, refers to any form of therapy which may affect vision, including, an agent which reduces or treats visual impairment in a subject, or an agent which results in visual impairment.

Agents, as identified using the methods of the invention include, but are not limited to, agents that are administered in vivo to the experimental vertebrate and agents that the eyes of the visually impaired experimental vertebrate may be exposed to for a suitable duration of time. Accordingly, agents that may be identified as being useful for treating or reducing visual impairment using methods of the invention may take the form of an agent that is administered to a subject, an agent that is topically applied to the eyes of the subject, or a light, such as, for example, in case of photodynamic therapy and laser based eye treatments. Exemplary agents include, but are not limited to, organic and inorganic compounds, peptides, vitamins, antioxidants, small interfering RNAs (siRNAs), photodynamic therapy, and laser treatment. Also, encompassed by the methods of the invention are agents that result in visual impairment.

The term “forelimb gait metrics,” as used herein, refers to the measurement of gait in a vertebrate, as it pertains to one or more forelimbs of the vertebrate. In one embodiment, forelimb gait metrics includes measurement of the forelimb braking duration. In another embodiment, the forelimb gait metrics includes measurement of forelimb stance variability. In yet another embodiment, forelimb gait metrics includes measurement of forepaw area during stance. In one embodiment, one or more of braking duration, forelimb stance variability and forepaw area during stance are measured as a measurement of forelimb gait metrics.

The term “upperlimb gait metrics,” as used herein, refers to the measurement of gait in a vertebrate, as it pertains to one or more upperlimbs of the vertebrate. In one embodiment, upperlimb gait metrics includes measurement of the upperlimb braking duration. In another embodiment, the upperlimb gait metrics includes measurement of upperlimb stance variability. In yet another embodiment, upperlimb gait metrics includes measurement of upperpaw area during stance. In one embodiment, one or more of braking duration, upperlimb stance variability and upperpaw area during stance are measured as a measurement of upperlimb gait metrics.

In one embodiment, a visually impaired subject shows a significant reduction in the braking duration of one or more forelimbs relative to the subject that is not visually impaired (i.e., control subject). In another embodiment, a visually impaired subject shows a significantly greater forelimb stance width variability relative to a control subject. In another embodiment, a visually impaired subject shows a significantly greater forepaw area during stance relative to the control subject. In yet another embodiment, a visually impaired subject shows one or more of a decrease in braking duration, an increase in forelimb stance width and an increase in forepaw area relative to a control subject.

In another embodiment, an experimental vertebrate shows one or more of: a significant reduction in the braking duration of one or more forelimbs; a significantly greater forelimb stance width variability; and a significantly greater forepaw area during stance in the presence of an agent relative to the braking duration, forelimb stance width variability and the forepaw area during stance in the absence of the agent.

In one embodiment, an experimental vertebrate having visual impairment is a vertebrate having at least one forelimb and showing a statistically significant decrease in braking duration of at least one forelimb, a statistically significant increase in stance width variability of at least one forelimb and a statistically significant increase in the area of at least one forepaw area during stance.

Accordingly, in one embodiment of the screening methods of the present invention, an experimental vertebrate having visual impairment is subjected to an agent for a suitable duration of time and the agent is identified as being useful in reducing or treating visual impairment if it results in having no statistically significant difference between the forelimb gait metrics of the experimental vertebrate and that of a control vertebrate which is not visually impaired. In one embodiment, an agent is identified as being useful for treating or reducing visual impairment in a subject if it results in having no statistically significant difference in one or more of braking duration, forelimb stance width variability and forepaw area during stance. Forelimb limb gait metrics including each of braking duration, forelimb stance width variability and forepaw area may be measured using any method known to one of skilled in the art, including methods known in the art and those described herein.

The experimental vertebrate that may be used in screening methods of the invention can be any vertebrate which includes at least one forelimb, and preferably at least three total limbs. Exemplary vertebrates useful in the methods described herein include, but are not limited to, rats, mice, hamsters, guinea pigs, cats, and dogs. In one embodiment, an experimental vertebrate useful in the methods of the invention is a rodent. Exemplary rodents that may be used in the screening methods of the invention include rats, mice, gerbils, hamsters, cavies, guinea pigs, and chinchillas.

Without wishing to be bound by theory, it is understood that any suitable means for the measurement of gait may be used in the methods of the invention. For example, in one embodiment, the apparatus can take the form of a gait imaging system, which includes a moveable belt track upon which a subject can ambulate. In one embodiment, the imaging system includes one or more imaging devices for recording the gait of an ambulating subject on the belt track. In one embodiment, an imaging device is disposed below the belt track to record contact between at least one forelimb of the subject and the belt track. However, it is understood that one or more imaging devices could be disposed anywhere with respect to the belt track, as long as such devices are able to record the gait of a'subject ambulating on the belt track. The subject can ambulate along the belt track in a substantially stationary location above the imaging device as the belt track moves, and the imaging device can record the contact by the subject.

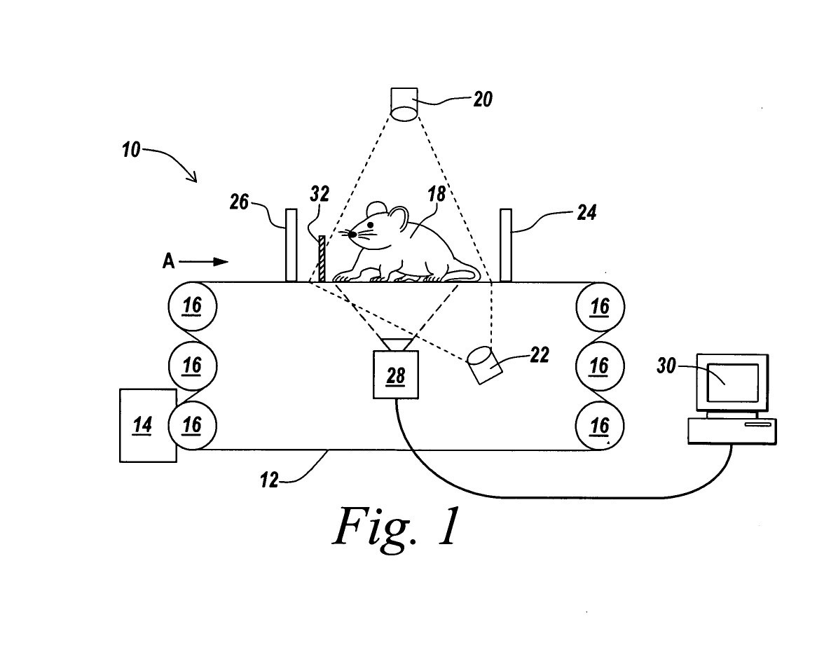

An exemplary gait measuring system that can be used in the screening methods of the invention is shown in FIG. 1. In one embodiment, the gait measuring system 10 includes a moveable belt 12 that weaves between a plurality of rotating drums, cogs, or wheels 16. The wheels 16 enable the belt 12 to move in a circumnavigating motion. In one embodiment shown in FIG. 1, the belt 12 is substantially transparent or translucent. A motor 14 couples to at least one of the wheels 16, forming a driving wheel for providing the motion to the belt 12. At least one experimental vertebrate, for example, a mouse 18 can ambulate along the belt 12 as described herein. In one embodiment, an image capturing device 28 is disposed beneath the belt 12. The image capturing device can take the form of one or more of a camera, a video camera, a digital camera, a camcorder, a digital camcorder, a digital image capture device and the like. The image capturing device 28 may also take the form of an ink pad, a touch pad, or Other pressure sensing pad. In one embodiment, at least one light 20 is disposed above the ambulating mouse 18. In another embodiment, a second light 22 can shine from beneath the belt track, which is generally translucent or transparent. In the exemplary gait measuring system depicted in FIG. 1, a back partition 24 and a front partition 26 prevent the mouse 18 from escaping and/or running faster than the speed of the belt. Also included are one or more impediments 32 disposed along the belt track between the back partition 24 and the front partition 26. In one embodiment, the impediments are disposed vertically with respect to the belt track. In another embodiment (not shown), the belt includes one or more visual cues disposed along the belt. Examples of visual cues include, but are not limited to, color, light, pattern, and shape. In one embodiment, one or more impediments include a color, a pattern or a shape. A computing device 30 can be in communication with the image collecting device 28, which enables a user of the apparatus 10 to control the acquisition of the images of the ambulating mouse 18 via the image collecting device 28.

The following examples provide illustrative embodiments of the present invention. One of ordinary skill in the art will recognize the numerous modifications and variations that may be performed without altering the spirit and scope of the present invention. Such modifications and variations are encompassed within the scope of the invention. The examples do not in any way limit the present invention.

### EXAMPLE 1

### Quantitative Measurement of Gait Reveals Differences in Gait Metrics Between Normal and Visually Impaired Mice

Analysis of gait via ventral plane videography was used to obtain a quantitative metrics of gait, which was different in some aspects between normal mice and mice that were visually impaired. In an exemplary experiment, gait was investigated in a total of 10 mice, 5 of whom had their eyes removed when the mice were about 8 weeks old. Each of the ten mice were made to walk on a transparent treadmill belt, and gait was assessed via ventral plane videography, as discussed herein and in an issued U.S. Pat. No. 6,899,686, the entire contents of which are incorporated by reference herein.

Visually impaired mice, i.e., mice with their eyes removed, were matched in age, size and gender with control mice. Both sets of mice were made to walk on a treadmill belt at the same speed and gait analysis was performed. The hindlimb and forelimb gait metrics were pooled as a measurement of gait metrics. As shown in Table 1, there was no significant difference in gait metrics between visually impaired mice and control mice. However, when the forelimb gait metrics were evaluated separately from the hind limb gait metrics, significant differences in gait metrics in the visually impaired mice and control mice were observed.

TABLE 1
Directional changes in gait metrics in visually
impaired mice as compared to control mice.

| | Hind and Forelimb | Hind Limbs | Forelimb |
|---|---|---|---|
| Gait Index | Pooled | Only | Only |
| Stride Length | 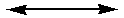 | 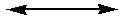 | 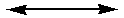 |
| Stride Frequency | 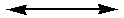 | 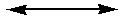 | 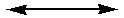 |
| % Swing | 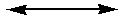 | 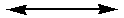 | 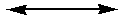 |
| % Braking | 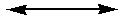 | 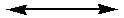 | 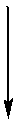 |
| % Propulsion | 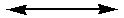 | 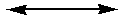 | 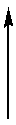 |
| Stance Width CV % | 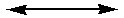 | 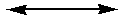 | 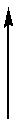 |
| Paw Area | 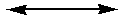 | 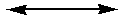 | 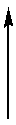 |

Whereas, when only gait metrics from hindlimbs were compared between the two sets of mice, the analysis of data did not indicate any differences in gait metrics between the visually impaired mice and control mice (see Table 1 and Table 2).

### EXAMPLE 2

### Forelimb Gait Metrics are Altered in Visually Impaired Mice

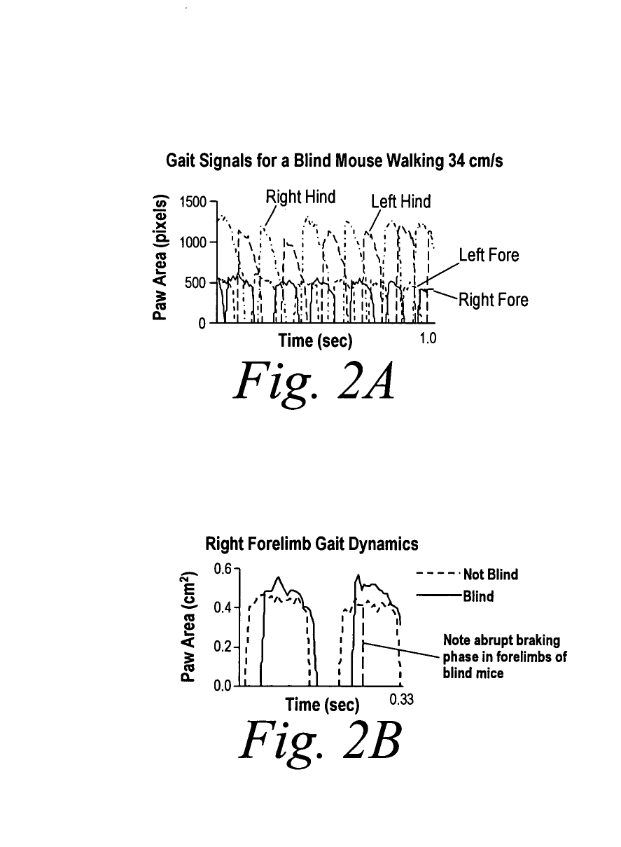

As indicated in Table 1, forelimb gait metrics were altered in mice that were visually impaired. In particular, the visually impaired mice showed a significant reduction in the braking duration of the forelimb gait metrics relative to those of the control mice, suggesting a role for vision in deceleration of the forelimbs during walking. Moreover, forelimb stance width variability was significantly greater in visually impaired mice relative to control mice, suggesting the importance of vision in maintaining balance during locomotion. This is especially notable since stance width variability increases have been associated with loss of balance and falls in a variety of medical conditions. Additionally, the fore paw area during stance was greater in the visually impaired mice compared to control mice. Interestingly, although the area of the fore paws of the visually impaired mice was comparable, both structurally and morphologically, to those of the control mice, the visually impaired mice exposed more area of the planar surface of their paws to contact the walking substrate during stance. This indicates the role of vision in proprioception of their forelimb or upper limbs during walking. The results are summarized in FIGS. 2A and 2B and Table 2.

TABLE 2
Gait dynamics in control mice and visually impaired mice
walking on a treadmill belt at a speed of 24 cm/s.

| | | Visually | |
|---|---|---|---|
| | Control | impaired | |
| | (sighted) | (blinded) | P |
| Measurement | N = 5 | N = 5 | value |
| Stride Length (cm) | 6.8 ± 0.1 | 6.7 ± 0.2 | 0.281 |
| Stride Frequency (Hz) | 4.9 ± 0.1 | 4.9 ± 0.2 | 0.348 |
| Stride Duration (ms) | 213 ± 4 | 208 ± 3 | 0.262 |
| Stance Duration (ms) | 139 ± 4 | 135 ± 3 | 0.317 |
| Swing Duration (ms) | 74 ± 3 | 73 ± 2 | 0.838 |
| Forelimb Stance Width (cm) | 1.72 ± 0.09 | 1.84 ± 0.07 | 0.305 |
| Forelimb Stance Width | 16.5 ± 2.2 | 30.0 ± 4.7 | <0.05 |
| Variability (%) | | | |
| Forelimb braking duration (ms) | 53 ± 1 | 40 ± 3 | <0.001 |
| Forelimb paw area (cm²) | 0.44 ± 0.01 | 0.55 ± 0.05 | <0.01 |
| Hind limb Stance Width (cm) | 2.64 ± 0.10 | 2.84 ± 0.09 | 0.176 |
| Hind limb Stance Width | 6.8 ± 0.2 | 9.2 ± 1.7 | 0.211 |
| Variability (%) | | | |
| Hind limb propulsion | 124 ± 4 | 122 ± 3 | 0.661 |
| duration (ms) | | | |
| Hind limb paw area (cm²) | 0.98 ± 0.05 | 1.1 ± 0.04 | 0.149 |

Means ± SE.

### EXAMPLE 3

### Assay for Measuring Effect of Vision on Gait

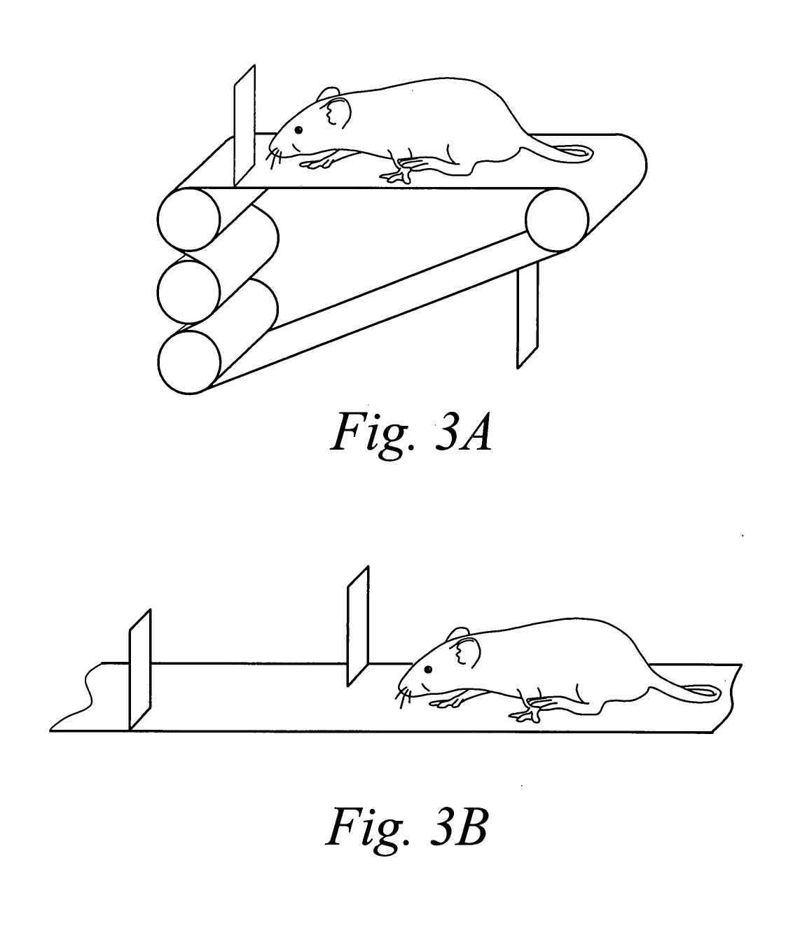

To further quantify the effect of vision on gait, a barrier or impediment was introduced into the path of the mice walking on the treadmill belt, as depicted in FIGS. 3A and 3B.

The impediment was configured such that it can plastically conform and lay flat against the treadmill belt should the impediment engage mechanical rollers during the revolution of the belt to which the impediment is attached, as shown in FIG. 3A.

The impediment may also be hinged so it moves flexibly when it encounters the animals or other objects in its path. The height, width, and colors of the impediment can be altered to investigate color, size, and depth perception. In some embodiments, a pattern can also be formed on the impediment.

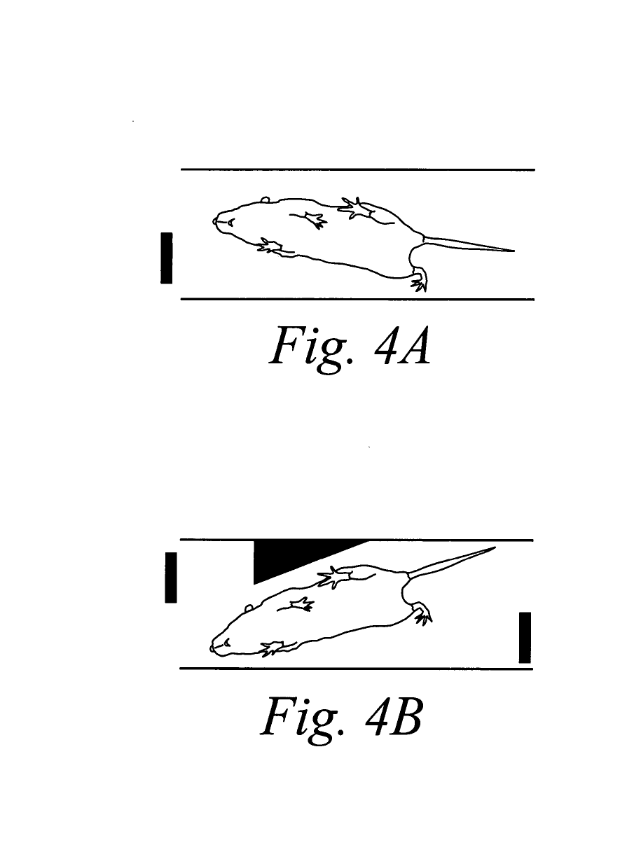

The ventral plane of a subject (in this instance mice), including its paws that are cycling through different aspects of stride, and the ventral view of the impediment can be viewed simultaneously with one camera, as depicted in FIGS. 4A and 4B.

Detection of alterations in gait can be synchronized with accurate determination of the impediment to walking such that determinations of the animal's ability to see or detect the impediment can be made. For example, if an animal and the impediment collide, it might be reasonable to conclude that the animal could not see the impediment as it approached the animal walking on the treadmill belt. Alternatively, if an animal's gait changes, for example, when a brightly red colored impediment approached the walking animal, it might be reasonable to conclude that the animal was able to see and detect bright red.

Additionally, an experimental subject ambulating on a belt track may also be exposed to a visual cue such as, for example, a pattern, a light, a color, light intensity, etc. Accordingly, the effect of one or more visual cues on forelimb gait metrics can be measured using the assays described herein. It is understood that a visual cue may either be affixed along the belt track, for example, present on an impediment disposed along the belt track, or it may be disposed any where within the line of vision of the ambulating experimental vertebrate.

The specification is most thoroughly understood in light of the teachings of the references cited within the specification which are hereby incorporated by reference. The embodiments within the specification provide an illustration of embodiments of the invention and should not be construed to limit the scope of the invention. The skilled artisan readily recognizes that many other embodiments are encompassed by the invention. All publications and patents, patent publications and non-patent references cited in this disclosure are incorporated by reference in their entirety. The citation of any references herein is not an admission that such references are prior art to the present invention.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may very depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

What is claimed is:

1. A method of identifying an agent which treats or reduces visual impairment in a subject comprising:
a) subjecting an experimental vertebrate having visual impairment to the agent; and
b) measuring the forelimb gait metrics of said experimental vertebrate;
wherein an alteration in the forelimb gait metrics in the presence of the agent relative to the forelimb gait metrics in the absence of the agent is indicative of the agent which treats or reduces visual impairment.

2. A method of identifying an agent which affects vision in a subject comprising:
a) subjecting an experimental vertebrate to the agent; and
b) measuring the forelimb gait metrics of said experimental vertebrate;
wherein an alteration in the forelimb gait metrics in the presence of the agent relative to the forelimb gait metrics in the absence of the agent is indicative of the agent which affects vision.

3. The method of claim 1, wherein the alteration comprises a decrease in a statistically significant difference between the forelimb gait metrics of the experimental vertebrate having visual impairment and a vertebrate not having visual impairment.

4. The method of claim 2, wherein an effect on vision comprises visual impairment.

5. The method of any one of claims 1 or 2, wherein subjecting comprises in vivo administration of the agent to the experimental vertebrate having visual impairment.

6. The method of any one or claims 1 or 2, wherein subjecting comprises exposing the eyes of the experimental vertebrate to the agent.

7. The method of any one of claims 1 or 2, wherein the agent is selected from the group consisting of an organic compound, an inorganic compound, a peptide, an antibody, an antioxidant, a vitamin, an siRNA, photodynamic therapy and laser treatment.

8. The method of any one of claims 1 or 2, wherein the experimental vertebrate comprises at least one forelimb.

9. The method of any one of claims 1 or 2, wherein the experimental vertebrate is a rodent.

10. The method of claim 9, wherein the rodent is selected from the group consisting of a mouse, a rat, a hamster, a chinchilla, a guinea pig, a cat and a dog.

11. The method of any one of claims 1 or 2, wherein the experimental vertebrate is placed on a moveable belt track for measuring the gait metrics.

12. The method of claim 11, wherein the belt track comprises one or more impediments disposed along the belt track.

13. The method of claim 11, wherein the belt track comprises one or more visual cues disposed along the belt track.

14. The method of claim 13, wherein the visual cues are selected from the group consisting of a pattern, a shape, a color and a light.

15. The method of any one of claims 12-14, wherein the gait metrics are measured using ventral plane videography.

16. The method of any one of claims 1 or 4, wherein the visual impairment is partial.

17. The method of any one of claims 1 or 4, wherein the visual impairment is complete.

18. The method of claim 1, wherein visual impairment is selected from the group consisting of blindness, cataract, myopia, color blindness, macular degeneration, cortical visual impairment, visuospatial disorientation, glaucoma, nystagmus, strabismus, retinoblastoma, retinopathy of prematurity, diabetic retinopathy, visual impairment associated with Parkinson's disease and visual impairment associated with multiple sclerosis.

19. A gait imaging system comprising:
(a) a moveable belt;
(b) one or more imaging devices disposed below or along the belt track; and
(c) one or more impediments and/or one or more visual cues disposed along the belt track.

20. The method of claim 19, wherein one or more impediments is affixed onto the belt track.

21. The gait imaging system of claim 19, wherein one or more impediments are flexibly hinged along the belt track.

22. The gait imaging system of claim 19, wherein one or more visual cues are disposed such that they are within the line of vision of a subject ambulating on the belt track.

23. The method of claim 19, wherein one or more imaging devices are selected from the group consisting of a camera, a camcorder, an image capturing device, and mirror reflect such images thereby captured.

24. A method of identifying an agent which treats or reduces visual impairment in a subject comprising:
a) subjecting an experimental vertebrate having visual impairment to the agent; and
b) measuring the upperlimb gait metrics of said experimental vertebrate;
wherein an alteration in the upperlimb gait metrics in the presence of the agent relative to the upperlimb gait metrics in the absence of the agent is indicative of the agent which treats or reduces visual impairment.

25. The method of claim 24, wherein the experimental vertebrate is a bipedal mammal.

26. The method of claim 25, wherein the bipedal mammal is a human.
